(19) United States
(12) Patent Application Publication
(10) Pub. No.: US 2023/0139600 A1
(43) Pub. Date: May 4, 2023

(54) ULTRAVIOLET RADIATION CLEANING SYSTEM FOR BAGGAGE TRAYS IN AN AIRPORT SECURITY ENVIRONMENT

(71) Applicants: Grinnory IP B.V., Capelle aan den IJssel (NL); One Tray S.P.A., Milan (IT)

(72) Inventors: Klaas van Rijn, Capelle aan den IJssel (NL); Age Durk Groenhof, Capelle aan den IJssel (NL); Alessandro Paolo Pedote, Milan (IT); Federico Maria Kluzer, Milan (IT)

(21) Appl. No.: 18/146,849

(22) Filed: Dec. 27, 2022

Related U.S. Application Data
(63) Continuation of application No. PCT/EP2022/054233, filed on Feb. 21, 2022.

(30) Foreign Application Priority Data
Mar. 8, 2021 (NL) 2027720

(51) Int. Cl. A61L 2/10

(52) U.S. Cl. CPC A61L 2/10; A61L 2202/14; A61L 2202/23; A61L 2202/122

(57) ABSTRACT

An ultraviolet radiation cleaning system for baggage trays in an airport security environment, comprising a radiation tunnel with an input for dirty trays and an output for clean trays, wherein at the input a first buffering zone is provided for a stack of dirty trays, and at the output a second buffering zone is provided for a stack of clean trays.

### CROSS-REFERENCE TO RELATED APPLICATIONS

This application is a continuation application of International Patent Application No. PCT/EP2022/054233, titled “AN ULTRAVIOLET RADIATION CLEANING SYSTEM FOR BAGGAGE TRAYS IN AN AIRPORT SECURITY ENVIRONMENT”, filed on Feb. 21, 2022, which claims priority to and the benefit of Netherland Patent Application No. 2027720, titled “AN ULTRAVIOLET RADIATION CLEANING SYSTEM FOR BAGGAGE TRAYS IN AN AIRPORT SECURITY ENVIRONMENT”, filed on Mar. 8, 2021, and the specification and claims thereof are incorporated herein by reference.

### BACKGROUND OF THE INVENTION

### Field of the Invention

The invention relates to an ultraviolet radiation cleaning system for baggage trays in an airport security environment, comprising a radiation tunnel with an input for dirty trays and an output for clean trays. The ultraviolet radiation cleaning system of the invention is intended to quickly and effectively disinfect the baggage trays that are used for security checks, and to efficiently and reliably eliminate micro-organisms such as bacteria, fungi and viruses from these trays. The term ‘quickly’ relates to the cleaning process taking less than about 30 seconds. The term ‘effectively’ means that after processing the trays, the amount of microorganisms on the trays have been removed for more than about 99% of the original quantity at the infeed of the cleaning system.

Existing solutions for cleaning trays with ultraviolet radiation in a passenger security process involve processing trays sequentially. A tray is put on a belt or rollers and goes through a tunnel and comes out clean on the other side. Only when a cleaned tray is taken from the belt or rollers, there is room to deliver the next cleaned tray. The problem with existing solutions is that picking up trays can be done at a faster pace than cleaning trays. The actual situation is even worse, taking into account that a passenger needs on average two trays. In addition, the existing solution requires a labour-intensive process to ensure a sufficient supply of trays to be cleaned. The level of experience of the responsible personnel in timely placing of trays to be cleaned, is at the end of the day decisive for limiting the idle time of the UVC cleaning tunnel.

Another disadvantage of existing solutions is that if a passenger picks up a tray, he or she has to wait until the next tray has been cleaned before being able to proceed with two clean trays. Some existing solutions offer the possibility to place two cleaned trays on the outlet belt from the UVC cleaning tunnel. Even then however, a waiting time (idle time) is created for the next passenger, which reduces the flow rate of the security process. This means that a higher than normal number of security setups has to be deployed due to inefficient cleaning. If the maximum capacity of security setups is used, this will consequently lead to waiting times and queues for the actual security process.

The invention is intended to provide a solution for the above-mentioned problems.

WO2021/022053 discloses a device for disinfecting a container, said device comprising: a housing having an upstream opening, a downstream opening, and an interior; a pathway extending between the upstream opening and the downstream opening; and a light source positioned in proximity to the pathway, wherein the light source is configured to emit an antimicrobial wavelength.

Note that this specification refers to various references. Discussion of such references is for a more complete background and is not to be construed as an admission that such references are prior art for patentability purposes.

### BRIEF SUMMARY OF THE INVENTION

According to an embodiment of the present invention, an ultraviolet radiation cleaning system for baggage trays in an airport security environment comprises the features of one or more of the appended claims.

In a first aspect of the invention, at the input of the cleaning tunnel a first buffering zone is provided for a stack of dirty trays, and at the output of the cleaning tunnel a second buffering zone is provided for a stack of clean trays. Accordingly, the invention makes cleaning of trays possible by UVC lighting in an airport security environment, whereby a continuously replenished stack/batch of trays can be processed, by destacking the trays, cleaning the trays individually and then stacking the trays at the output buffering zone. As will be explained hereinafter there are several options to stack the trays at the output buffering zone.

Preferably a destacking system is provided at the input for removal of an individual dirty tray from the stack of dirty trays in the first buffering zone, and a first transfer mechanism to receive said individual dirty tray from the destacking system and to supply said individual dirty tray taken from the stack of dirty trays to the radiation tunnel.

Accordingly, it is also preferred that a transfer mechanism is provided at the output to transfer an individual cleaned tray from the radiation tunnel to a stacking system for receiving such individual cleaned tray and to supply that individual cleaned tray to the stack of clean trays in the second buffering zone.

Suitably the destacking system and/or the stacking system comprises four tray handling organs that are operative near corner points of the trays. In this way the handling of the trays can be carried out efficiently and reliably.

Each tray handling organ can be effective and yet be provided at low cost, when it comprises two flanges that share a common vertical axis, wherein the said two flanges are jointly rotatable around said vertical axis, and wherein said two flanges define a slit between the two flanges which is equipped to receive an edge from a tray to be lifted or lowered, wherein the two flanges have obliquely oriented surfaces that are facing each other and that delimit the slit for the edge of the tray so as to arrange that rotation of the tray handling organ will cause the slit to change in height. Accordingly, the tray that is handled will be correspondingly lifted or lowered.

In a first embodiment of the system of the invention, the second buffering zone at the output is arranged to receive and add an individual cleaned tray to the bottom of the stack of cleaned trays.

Desirably the first embodiment is arranged such that the second buffering zone at the output is equipped with a first sensor for monitoring the height of the stack of cleaned trays.

In a second embodiment of the system of the invention, the second buffering zone at the output is arranged to receive and add an individual cleaned tray to the top of the stack of cleaned trays.

Desirably the second embodiment is arranged such that the system is provided with a control system and a second sensor to monitor the altitude of the top of the stack of cleaned trays so as to maintain this altitude.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings

FIGS. 1A, 1B, 1C and 1D are illustrations from a transparent side view, a side view, a frontal view and a top view respectively of a first system according to an embodiment of the present invention;

FIGS. 2A, 2B and 2C are illustrations of a system according to an embodiment of the present invention in an airport security environment;

FIG. 3 is an illustration of a single tray handling organ to be placed near a corner point of a tray or trays to be handled according to an embodiment of the present invention;

FIGS. 4A, 4B, 4C, 4D, 4E, 4F, 4G, 4H, 4I, and 4J are illustrations showing operation of the first system according to an embodiment of the present invention;

FIGS. 5A and 5B are illustrations from a transparent side view and a side view respectively of a second embodiment of a system according to the invention;

FIGS. 6A, 6B and 6C show three variations on the second embodiment of the system of the invention; and

FIGS. 7A, 7B, 7C, 7D, 7E, 7F, 7G, 7H, 7I, 7J, 7K, 7L, 7M, 7N, 7O, 7P, 7Q, 7R, 7S, 7T, 7U, 7V, 7W, 7X, 7Y and 7Z are illustrations showing operation of the second embodiment of the system of the invention.

Note that, while the above refers to a “first” and “second” embodiment, such should not be interpreted to mean that the first embodiment is a preferred embodiment or that there are only two embodiments of the present invention. References to a “first” and “second” embodiment is simply for ease of describing variations of the present invention.

### DETAILED DESCRIPTION OF THE INVENTION

Whenever in the figures the same reference numerals are applied, these numerals refer to the same parts.

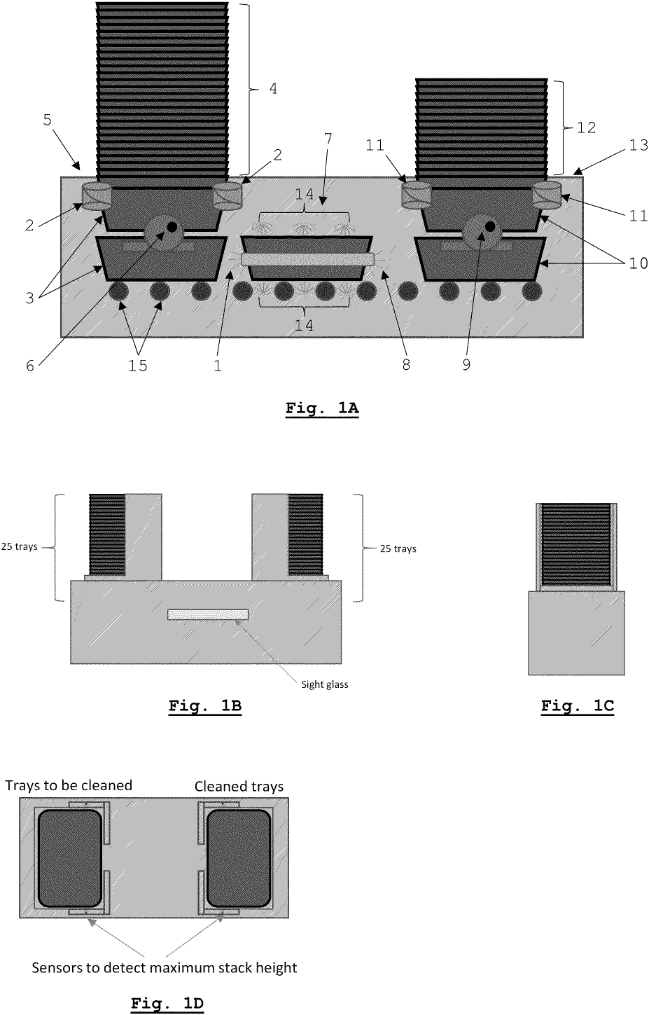

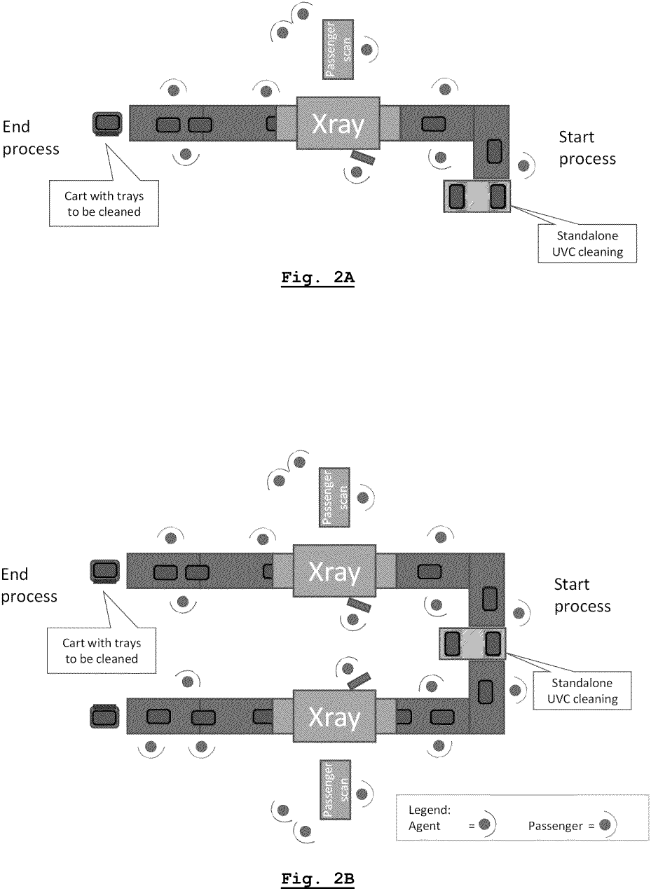

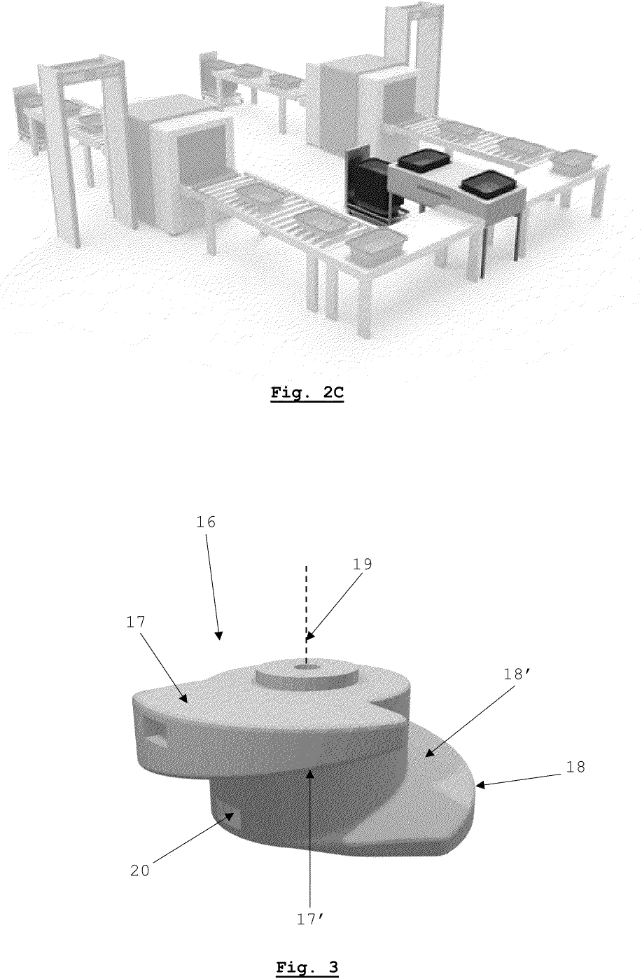

FIGS. 1-4 relate to a first embodiment of the ultraviolet radiation cleaning system of the invention. FIGS. 1A-1D show the ultraviolet radiation cleaning system for baggage trays in an airport security environment as depicted in FIGS. 2A-2C, comprising a radiation tunnel 7 with an input 1 for dirty trays and an output 8 for clean trays 10, wherein the input 1 is equipped with a first buffering zone 5 for a stack 4 of dirty trays, and the output 8 is equipped with a second buffering zone 13 for a stack 12 of clean trays.

It is schematically shown in FIGS. 1A-1D that at the input 1 a destacking system 2 is provided for removal of an individual dirty tray 3 from the stack 4 of dirty trays in the first buffering zone 5, and a first transfer mechanism 6 to receive said individual dirty tray 3 from the de-stacking system 2 and to supply said individual dirty tray 3 taken from the stack 4 of dirty trays to the radiation tunnel 7.

It is further shown in FIGS. 1A-1D that at the output 8 a transfer mechanism 9 is provided to transfer an individual cleaned tray 10 from the radiation tunnel 7 to a stacking system 11 for receiving such individual cleaned tray 10 and to supply that individual cleaned tray 10 to the stack 12 of clean trays in the second buffering zone 13.

It is remarked that the destacking system 2 and/or the stacking system 11 comprises four tray handling organs 16 that are operative near corner points of the trays 3, 10. One tray handling organ 16 is shown in FIG. 3. This FIG. 3 shows that such a tray handling organ 16 comprises two flanges 17, 18 that share a common vertical axis 19, wherein the said two flanges 17, 18 are jointly rotatable around said vertical axis 19. The two flanges 17, 18 define a slit 20 between the two flanges 17, 18 which is equipped to receive an edge from a tray 3, 10, wherein the two flanges 17, 18 have obliquely oriented surfaces 17′, 18′ that are facing each other and that delimit the slit 20 to receive an edge of the tray 3, 10 so as to arrange that rotation of the tray handling organ 16 will cause the slit 20 to change in height. Accordingly, also the tray 3, 10 will be changed in height.

Particular for this first embodiment of the ultraviolet radiation cleaning system of the invention as depicted in FIGS. 1-4, is that the second buffering zone 13 at the output 8 is arranged to receive and add an individual cleaned tray 10 to the bottom of the stack 12 of cleaned trays.

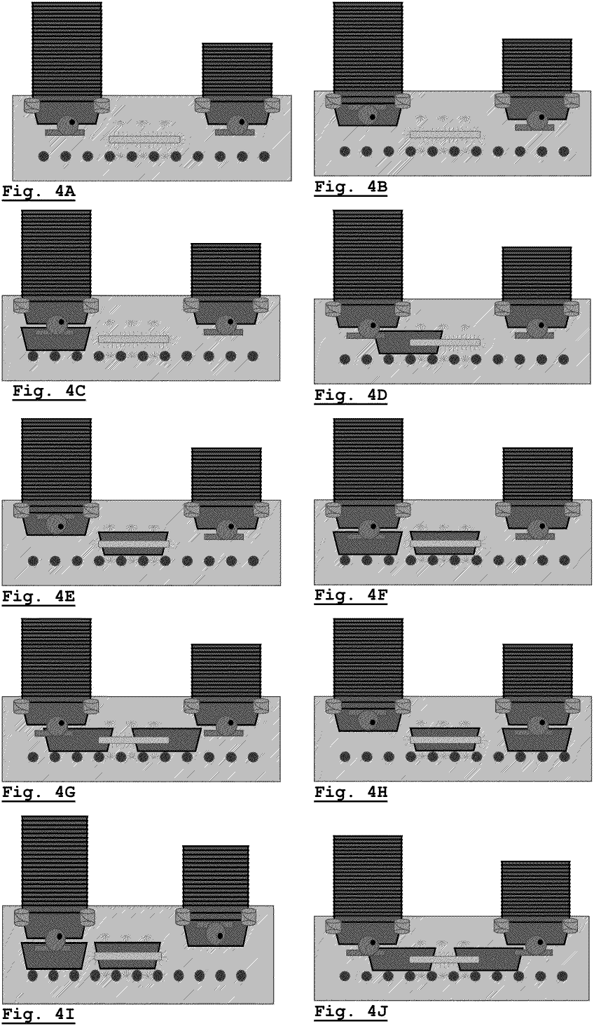

The operation of this first embodiment of the ultraviolet radiation cleaning system of the invention, will be further explained hereinafter with reference to FIGS. 4A-4J.

FIG. 4A: Supply of trays. Trays are provided on the stack 4 at the input buffering zone 5.

There is always one tray in the tray handling organs 2 on the input side and one tray in the tray handling organs 11 on the output side. This is to prevent a passenger or coworker from getting their hands/arms into the cleaning system. Furthermore, the tray in the tray handling organs 2, 11 serves to shield the UVC light of the lamps 14. If the stack 4 of trays is too high, the tray handling organs 2 will stop and a warning light will flash. The detection of whether the stack 4 is too high can be determined with a sensor.

FIG. 4B: Destacking. The stack 4 at the infeed is destacked per tray by means of four tray handling organs 2, each with its own motor. The tray 3 lands on lift arms of a lift mechanism 6.

FIG. 4C: Controlled placement of tray on rollers. The lift mechanism 6 places the tray 3 on the driven rollers 15.

FIG. 4D: Cleaning situation 1. The tray 3 is transported by the driven rollers 15 and cleaned with UVC light from the lamps 14. The cleaning of the tray 3 starts as soon as it is within reach of the lamps 14 and ends when the tray 3 has been transported from the lamps 14 to the output 8.

FIG. 4E: Cleaning situation 2. The tray 3 is transported by the driven rollers 15 and cleaned with UVC light at the bottom, side and top (360 degrees, all sides). The speed of transport of the tray 3 is adjusted to the required UVC exposure time to clean the tray at least 99.9%. The tray handling organs 2 at the input buffering zone 5 place the next tray in the transfer mechanism 6.

FIG. 4F: controlled placement of a next tray on the rollers 15 while another tray is being exposed to UVC lighting. The transfer mechanism 6 places the next tray on the driven rollers 15, while the previous tray is illuminated with UVC light.

FIG. 4G: Transport and lighting. The tray that is transported from the lighting position is still illuminated as long as it is within range of the UVC lamps 14. The next tray is transported and for this purpose the lighting starts as soon as it is in the range of the UVC lamps 14.

FIG. 4H: Position tray in stacking mechanism. The edge of the cleaned tray 10 is above the guides of the stacking mechanism 11 at the output buffering zone 13.

FIG. 4I: stacking mechanism. By means of the transfer mechanism 9, the tray 10 is placed in the tray handling organs 11 that take over the tray 10 for stacking up.

FIG. 4J: Stack up. The tray which was still in the tray handling organs 11 is stacked upwards by the tray handling organs 11 with the upward movement of the tray. The tray 10 remains in the tray handling organs 11 until the next tray is raised, so as to provide that on the one hand access from below is prevented so that passengers and employees cannot enter it with their hand or arm, and on the other hand to shield against UVC light.

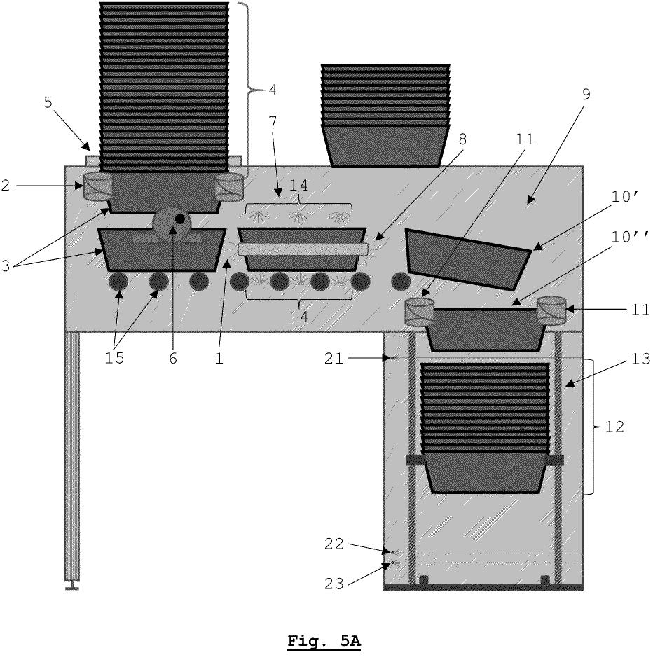

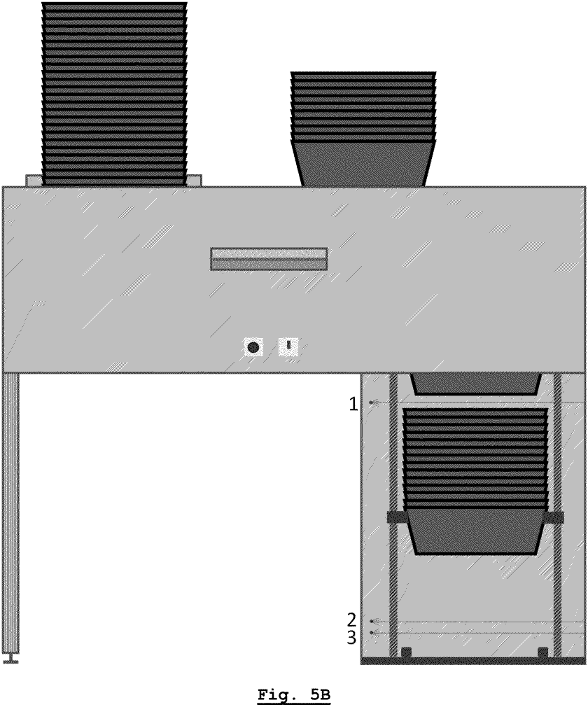

FIGS. 5-7 relate to a second embodiment of the ultraviolet radiation cleaning system of the invention. FIGS. 5A-5D show the ultraviolet radiation cleaning system for baggage trays, which, similar to the first embodiment of FIGS. 1-4, can be applied in an airport security environment as depicted in FIGS. 2A-2C. The cleaning system according to the second embodiment also comprises a radiation tunnel 7 with an input 1 for dirty trays 3 and an output 8 for clean trays 10, wherein the input 1 is equipped with a first buffering zone 5 for a stack 4 of dirty trays, and the output 8 is equipped with a second buffering zone 13 for a stack 12 of clean trays.

It is schematically shown in FIGS. 5A-5B that at the input 1 a destacking system 2 is provided for removal of an individual dirty tray 3 from the stack 4 of dirty trays in the first buffering zone 5, and a first transfer mechanism 6 to receive said individual dirty tray 3 from the destacking system 2 and to supply said individual dirty tray 3 taken from the stack 4 of dirty trays to the radiation tunnel 7.

It is further shown in FIGS. 5A-5B that at the output 8 a transfer mechanism 9 is provided to transfer an individual cleaned tray 10 from the radiation tunnel 7 to a stacking system 11 for receiving such individual cleaned tray 10 and to supply that individual cleaned tray 10 to the stack 12 of clean trays in the second buffering zone 13.

Also, in this second embodiment the destacking system 2 and/or the stacking system 11 comprises four tray handling organs 16 that are operative near corner points of the trays. One tray handling organ 16 is shown in FIG. 3. FIG. 3 shows that such a tray handling organ 16 comprises two flanges 17, 18 that share a common vertical axis 19, wherein the said two flanges 17, 18 are jointly rotatable around said vertical axis 19. The two flanges 17, 18 define a slit 20 between the two flanges 17, 18 which is equipped to receive an edge from a tray 3, 10, wherein the two flanges 17, 18 have obliquely oriented surfaces 17′, 18′ that are facing each other and that delimit the slit 20 to receive an edge of the tray 3, 10 so as to arrange that rotation of the tray handling organ 16 will cause the slit 20 to change in height. Accordingly, also the tray 3, 10 will be changed in height.

Particular for this second embodiment of the ultraviolet radiation cleaning system of the invention as depicted in FIGS. 5-7, is that the second buffering zone 13 at the output 8 is arranged to receive and add an individual cleaned tray 10 to the top of the stack 12 of cleaned trays.

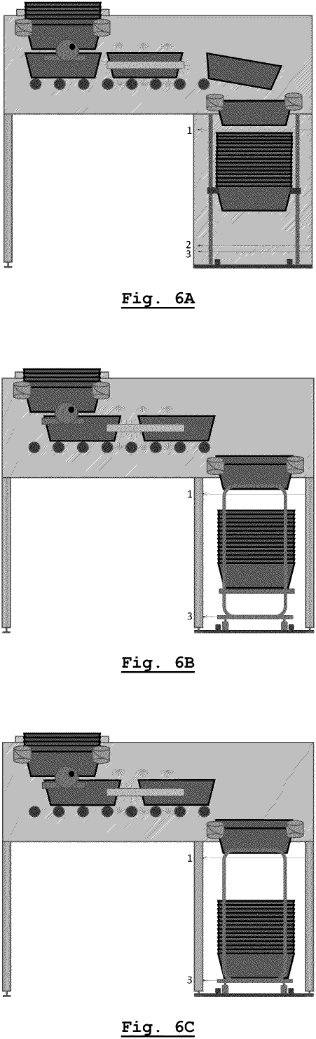

FIGS. 6A-6C show three variations to the second embodiment, to note:
in FIG. 6A: Transfer system which lowers stack. Fall distance of tray is short. System detects when a cart is positioned and then places trays on it.
in FIG. 6B: Lowerator slowly lowers due to weight of trays. Fall distance of trays is small.
in FIG. 6C: Cart where the trays fall onto. This works well because of the shape and nestability of the trays. One tray is attached to the cart to have a correct starting position.

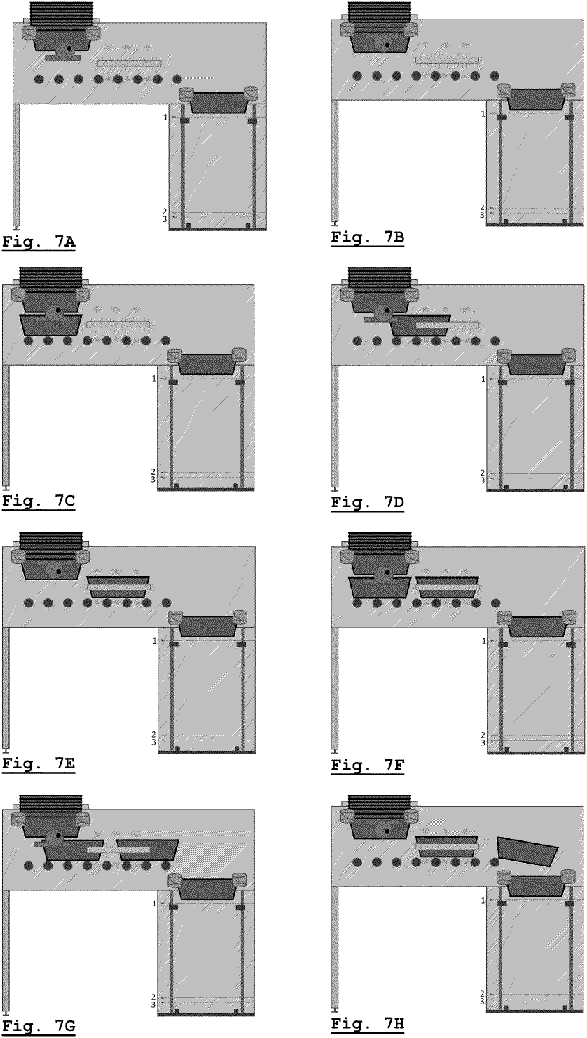

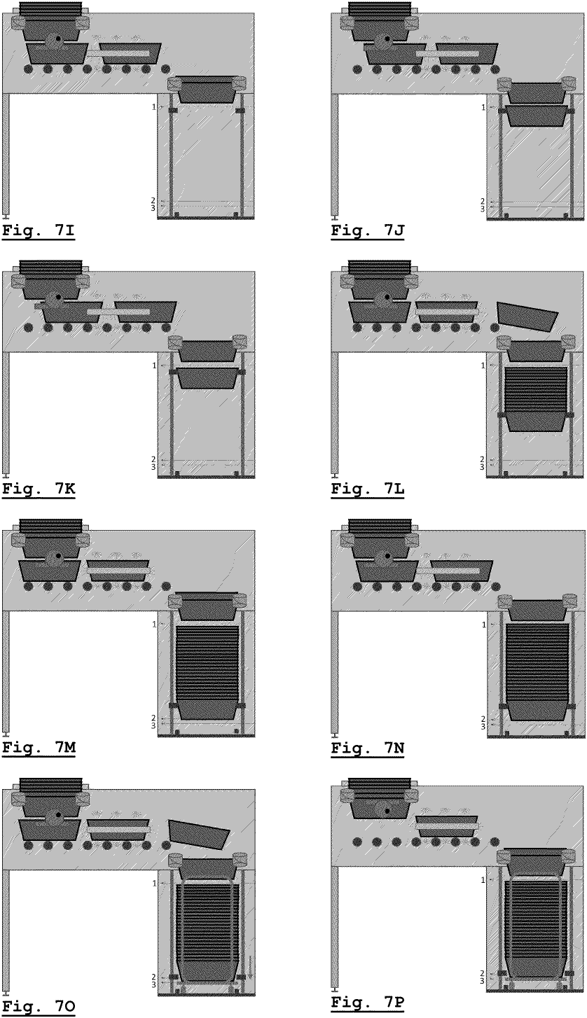

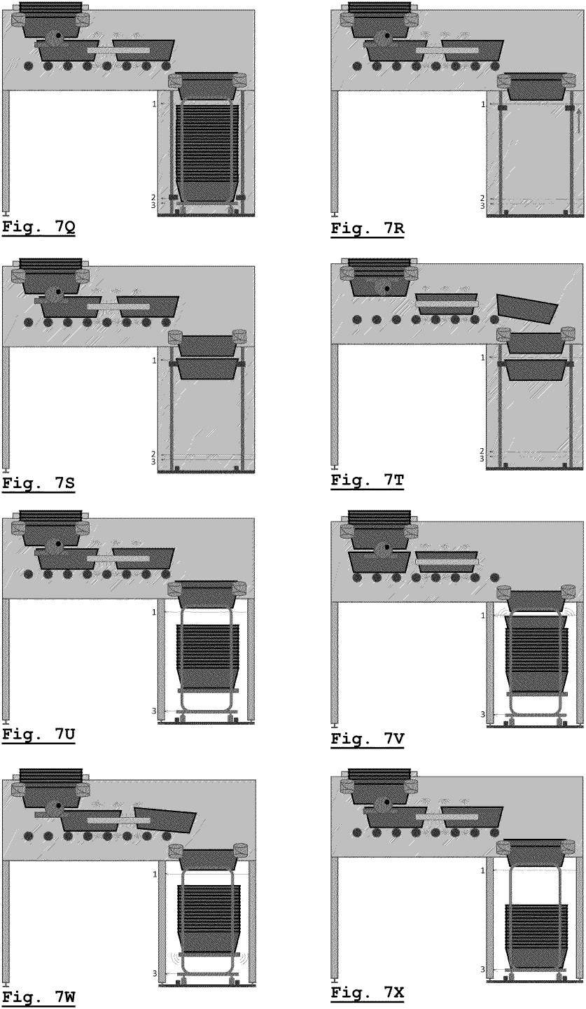

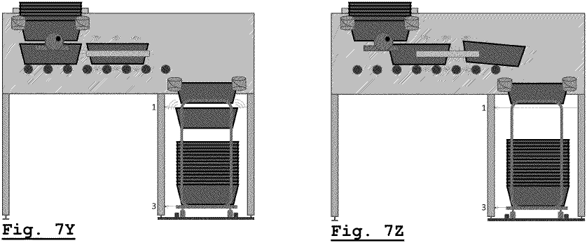

The operation of this second embodiment of the ultraviolet radiation cleaning system of the invention and the three mentioned variations to this embodiment, will be further explained hereinafter with reference to FIGS. 7A-7Z.

FIG. 7A: Supply of trays. Trays are provided on the stack 4 at the input buffering zone 5. There is always one tray in the tray handling organs 2 on the input side and one tray in the tray handling organs 11 on the output side. This is to prevent a passenger or coworker from getting their hands/arms into the cleaning system. Furthermore, the tray in the tray handling organs 2, 11 serves to shield the UVC light from the lamps 14. If the stack 4 of trays is too high, the tray handling organs 2 will stop and a warning light will flash. The detection of whether the stack 4 is too high can be determined with a sensor.

FIG. 7B: Destacking. The stack 4 at the input buffering zone 5 is destacked per tray by means of four tray handling organs 2, each with its own motor.

FIG. 7C: Controlled placement of tray on rollers 15. The transfer mechanism 6 places the tray 3 on the powered rollers 15.

FIG. 7D: Cleaning. The tray 3 is transported by the driven rollers 15 and cleaned with UVC light with lamps 14 at the bottom, side and top (360 degrees, all sides). The speed of transport of the tray 3 is adjusted to the UVC exposure time that is required to clean the tray at least 99.9%.

FIG. 7E: Clean and destacking the next tray. The tray handling organs 2 at the input buffering zone 5 place the next tray in the transfer mechanism 6.

FIG. 7F: Clean and place next tray. The transfer mechanism 6 places the next tray on the driven rollers 15, while the previous tray is still illuminated with UVC light.

FIG. 7G: Cleaning and transportation. The tray that is transported from the exposure position is still illuminated as long as it is within range of the UVC lamps 14. The next tray is transported and for this purpose the lighting starts as soon as it is in the range of the UVC lamps 14.

FIG. 7H: Stacking 1. The cleaned tray 10′ is thrown onto the cleaned tray 10″ in the tray handling organs 11 at the output buffering zone 13. This output buffering zone 13 also uses four tray handling organs, each with its own motor. There is always a tray in these four tray handling organs 11 at the output buffering zone 13, so as to close off access from below so that passengers and employees cannot enter it with their hand or arm, and on the other hand to shield against UVC light from the lamps 14.

FIG. 7I: Stacking 2. The cleaned tray 10′ lands on the tray 10″ which still is in the tray handling organs 11.

FIG. 7J: Destacking. The tray located in the tray handling organs 11 is destacked and lands on the lift system. The top edge of the tray is detected by a sensor 21.

FIG. 7K: Lift system-situation 1. The lift system lowers until sensor 21 is free from detection again.

FIG. 7L: Lift system-situation 2. Situation in which several trays are stacked and the lift system always drops a tray height, so that sensor 21 is free from detection.

FIG. 7M: Lift system-situation 3. Situation where the lift system is almost full. The stack has dropped into reach of sensor 22 and sensor 21 is still free from detection. There is still room for one tray.

FIG. 7N: Lift system-situation 4. Situation where the lift system is full. The stack is still detected by sensor 22 and now also by sensor 21. No more trays can be added (max 25 trays).

FIG. 7O: Lift system-situation 5. Situation where the lift system is full. A cart is placed. The lift system lowers and places the stack on the cart. The cart is detected by sensor 23. If a cart is placed before the lift system is full (and is detected by sensor 23), stacking will stop as soon as the lift system is detected by sensor 22. The lift system then places the trays on the cart.

FIG. 7P: Lift system-situation 6. If the lift system is full and the cart has not yet been taken out, the process will stop if there is a tray inside and a tray on the tray handling organs 11.

FIG. 7Q: Lift system-situation 7. The lift system is full, the cart has not been taken out yet and there is a tray inside and a tray on the tray handling organs 11. The rollers 15 stop when the tray reaches the last roller. The UVC exposure by lamps 14 stops. The rollers 15 and UVC lighting by lamps 14 start again when the process continues, so when the full cart has been removed.

FIG. 7R: Lift system-situation 8. The full cart has been removed. The lift system returns to the top to below sensor 21.

FIG. 7S: Lift system-situation 9. The UVC lighting starts again, the drive of rollers 15 starts again. The tray in the tray handling organs 11 is destacked and lands on the lift system. The tray is detected by sensor 21.

FIG. 7T: Lift system-situation 10. The lift system lowers until sensor 21 is free from detection again in accordance with the previous Lift system-situation 1.

FIG. 7U: Application of a lowerator. There are now two trays on the tray handling organs 11 at the output, through which the bottom tray can be destacked and stacked on the lowerator. Sensor 23 detects that there is a lowerator below the output. Sensor 22 is not in use with the lowerator.

FIG. 7V: Destacking. The bottom tray is destacked and falls on the lowerator.

FIG. 7W: Lowerator drops. Due to the weight of the tray, the lowerator drops a bit.

The moment the lowerator comes to its lowest position and a tray is added that remains detected by sensor 21, the stack is at maximum height and stacking stops. Other operation is identical with the operation when using a lift system.

FIG. 7X: Detection cart present. There are now two trays on the tray handling organs 11 at the output, allowing the bottom tray to be destacked and stacked on the cart.

Sensor 23 detects that there is a cart present at the output. Sensor 22 is not in use when applying a cart.

FIG. 7Y: Destacking. The bottom tray is destacked and falls on the cart. Even at a greater height, the tray falls into the bottom tray due to the nesting of the trays.

FIG. 7Z: Stack on cart. When a tray is added, the stack 12 increases. If a tray remains detected by sensor 21, the stack is at maximum height and stacking stops. One tray is attached to the cart to have a correct starting position. Other operation is identical to using a lift system.

Although the invention has been discussed in the foregoing with reference to an exemplary embodiment of the method of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

Note that in the specification and claims, “about” or “approximately” means within twenty percent (20%) of the numerical amount cited.

What is claimed is:

1. An ultraviolet radiation cleaning system for baggage trays in an airport security environment, the system comprising:
a radiation tunnel comprising an input for dirty trays and an output for clean trays;
a first buffering zone disposed at the input and equipped to receive a stack of dirty trays; and
a second buffering zone disposed at the output and equipped to receive a stack of clean trays.

2. The ultraviolet radiation cleaning system of claim 1, further comprising:
a destacking system disposed at the input for removal of an individual dirty tray from the stack of dirty trays in the first buffering zone; and
a first transfer mechanism to receive said individual dirty tray from the destacking system and to supply said individual dirty tray taken from the stack of dirty trays to the radiation tunnel.

3. The ultraviolet radiation cleaning system of claim 2, further comprising:
a second transfer mechanism disposed at the output to transfer an individual cleaned tray from the radiation tunnel to a stacking system for receiving such individual cleaned tray and to supply that individual cleaned tray to the stack of clean trays in the second buffering zone.

4. The ultraviolet radiation cleaning system of claim 3, wherein the destacking system and/or the stacking system comprises four tray handling organs that are operative near corner points of the trays.

5. The ultraviolet radiation cleaning system of claim 4, wherein each tray handling organ comprises two flanges that share a common vertical axis, wherein the two flanges are jointly rotatable around said vertical axis, and wherein said two flanges define a slit between the two flanges which is equipped to receive an edge from a tray, wherein the two flanges have obliquely oriented surfaces that are facing each other and that delimit the slit for the edge of the tray so as to arrange that rotation of the tray handling organ will cause the slit to change in height.

6. The ultraviolet radiation cleaning system of claim 3, wherein the second buffering zone at the output is arranged to receive and add an individual cleaned tray to the bottom of the stack of cleaned trays.

7. The ultraviolet radiation cleaning system of claim 6, wherein the second buffering zone at the output comprises a first sensor for monitoring the height of the stack of cleaned trays.

8. The ultraviolet radiation cleaning system of claim 3, wherein the second buffering zone at the output is arranged to receive and add an individual cleaned tray to the top of the stack of cleaned trays.

9. The ultraviolet radiation cleaning system of claim 8, the system further comprising a control system and a second sensor to monitor the altitude of the top of the stack of cleaned trays so as to maintain this altitude.
